(19) United States
(12) Patent Application Publication
(10) Pub. No.: US 2016/0287826 A1
(43) Pub. Date: Oct. 6, 2016

(54) MEDICO-SURGICAL TUBES

(71) Applicant: SMITHS MEDICAL INTERNATIONAL LIMITED, Ashford, Kent (GB)

(72) Inventor: Stephen James Field, Kent (GB)

(73) Assignee: SMITHS MEDICAL INTERNATIONAL LIMITED, Ashford, Kent TN25 4BF (GB)

(21) Appl. No.: 15/035,256

(22) PCT Filed: Oct. 16, 2014
(86) PCT No.: PCT/GB2014/000419

§ 371 Date: May 9, 2016

(30) Foreign Application Priority Data
Nov. 19, 2013 (GB) 1320393.0

(51) Int. Cl. A61M 16/04; A61M 16/08; A61M 16/20; A61M 39/08

(52) U.S. Cl. CPC A61M 16/0465; A61M 39/08; A61M 16/0434; A61M 16/0816; A61M 16/20; A61M 16/0463; A61M 16/0486; A61M 2205/02; A61M 2207/00

(57) ABSTRACT

A tracheostomy tube (1) is made from a first component (20) of a relatively hard plastics material that is overmoul - ded with a second component (29) of a softer plastics material (26). The first component (20) provides a machine end coupling (6) and a helical reinforcement member (23, 23′) extending along the region (3) that will, in use, extend through the tracheal tissue. The second component (29) extends along the reinforcement member (23, 23′) and beyond it to form the patient end (2) of the tube (1).

This invention relates to medico-surgical tubes of the kind adapted to extend through an opening into a body cavity.

The invention is more particularly, but not exclusively, concerned with tracheostomy tubes.

Tracheostomy tubes are used to enable ventilation or respiration of a patient. They are inserted into the trachea via a surgically-formed opening in the neck so that one end of the tube locates in the trachea and the other end locates outside the patient adjacent the neck surface. Tracheostomy tubes are generally used for more long-term ventilation or where it is not possible to insert an airway through the mouth or nose. Various types of different tracheostomy tubes are presently available to suit different needs. The tube may be reinforced along its length, such as with a helically wound metal wire, where there is a risk the tube might be crushed and occluded, such as might happen when the tube is flexed. Alternatively, the tube may be moulded of a soft material, such as a silicone plastics material, where there is a greater risk of trauma to the tracheal lining, such as in paediatric patients or patients with a damaged trachea. Tracheostomy tubes are also available with an inner cannula so that the inner cannula can be removed and replaced periodically to prevent the build-up of secretions and avoid the need to replace the tube itself. The wall of some tubes are formed with a fenestration or opening within the trachea by which air can flow to the vocal folds to enable speech. The variety of different types of tubes can make selecting the appropriate tube a problem especially in circumstances where the patient's condition may change after the tube has been inserted. Tracheostomy tubes can also be inserted by different techniques, such as the surgical cut down procedure carried out in an operating theatre or a percutaneous dilatation procedure, which may carried out in emergency situations. These different techniques ideally require different tracheostomy tubes specially adapted for the respective techniques.

It is an object of the present invention to provide an alternative medico-surgical tube.

According to one aspect of the present invention there is provided a medico-surgical tube of the above-specified kind characterised in that the tube is formed of at least first and second materials of different hardness, that the first material is harder than the second material, that the region of the tube extending within the opening has a greater proportion of the first material to the second material than the region of the tube extending within the body cavity such that the region of the tube extending within the opening is more rigid than the region of the tube extending within the body cavity.

The tube may have a coupling at its machine end formed primarily of the first material. The tube may have an elongate member of the first material extending along the region of the tube extending within the opening, the elongate member being embedded in the second material. The elongate member may extend around and along the region within the opening and may, for example, be of helical shape. The tube may have a flange formed by a member of the first material and an overmoulding of the second material. The member of the first material may include markings visible through the second material. The member of the first material may include markings visible through,the second material.

According to another aspect of the present invention there is provided a tracheostomy tube formed of a first, relatively hard material and a, second, relatively soft material, the tube having a machine end coupling located outside the body and formed substantially entirely of the first material, the tube having a portion extending through neck tissue and formed of a combination of the first and second materials, and the tube having a patient end portion extending within the trachea and formed substantially entirely of the second, relatively soft material.

Both the first and second materials are preferably plastics materials and may be different grades of the same type of plastics.

A tracheostomy tube and various modifications according to the present invention will now be described, by way of example, in which:

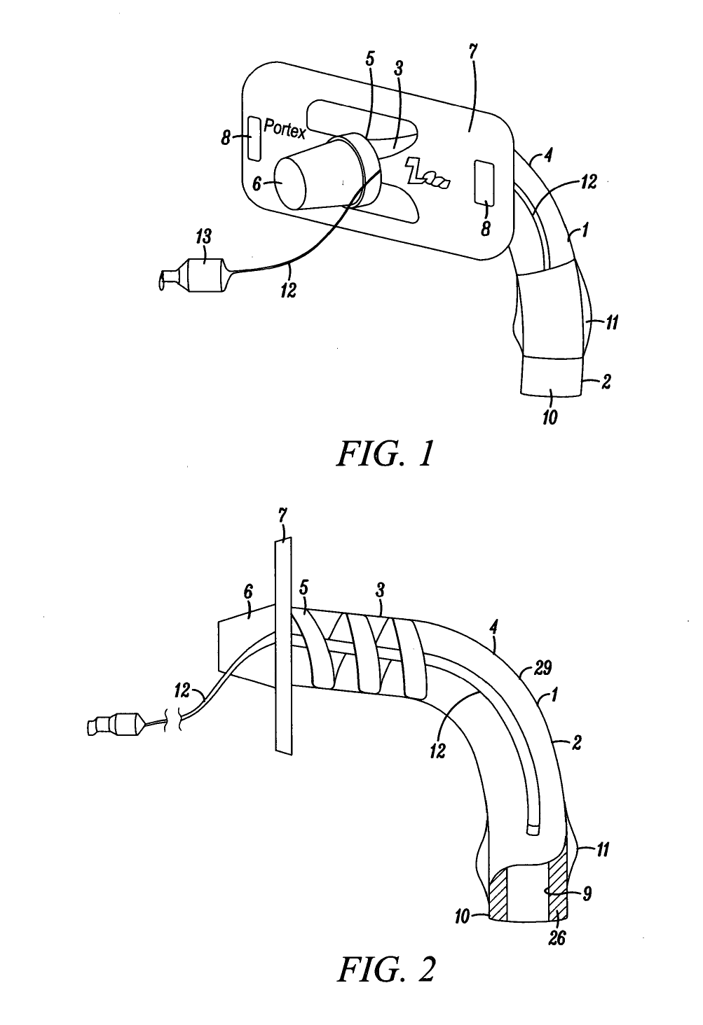

FIG. 1 is a perspective view of the tube;

FIG. 2 is a side elevation view of the tube partly in cross-section;

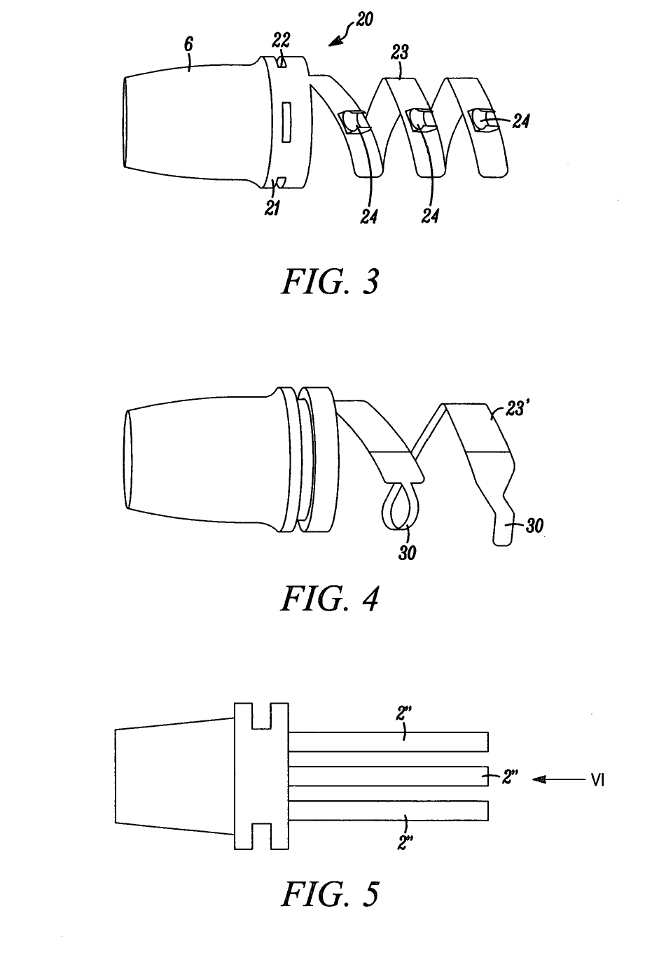

FIG. 3 is a side elevation view of a machine end component of the tube;

FIG. 4 is a perspective view of a modified form of the component shown in FIG. 3;

FIG. 5 is a side elevation view of an alternative version of the machine end component;

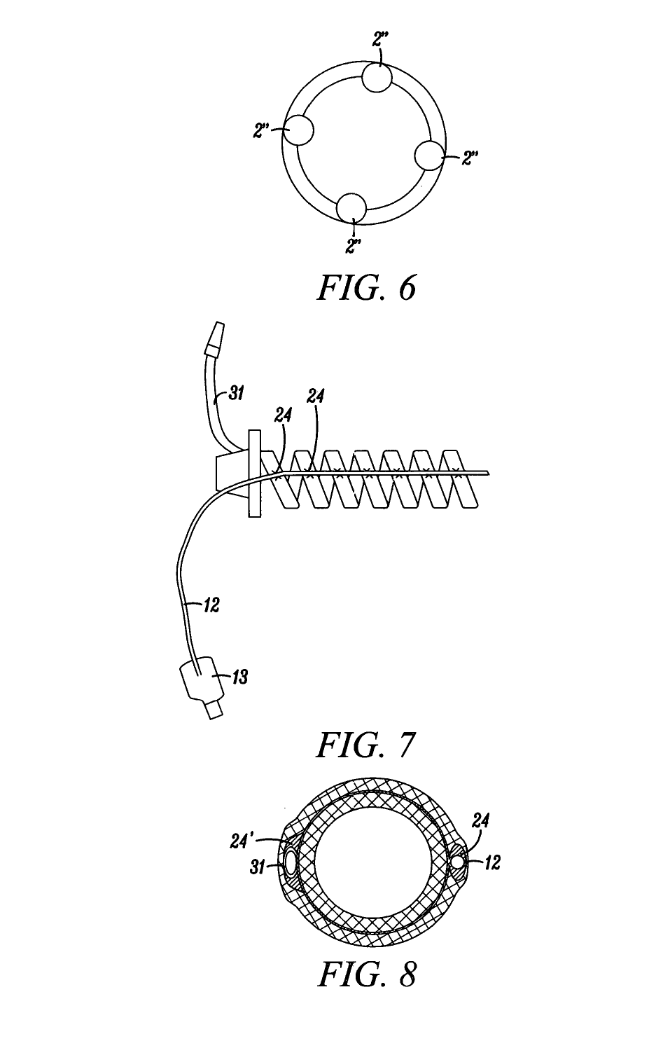

FIG. 6 is an end view of the component shown in FIG. 5 along the arrow VI;

FIG. 7 is a side elevation view of a machine end component with a suction line;

FIG. 8 is a transverse cross-section view of a tube including the component shown in FIG. 7;

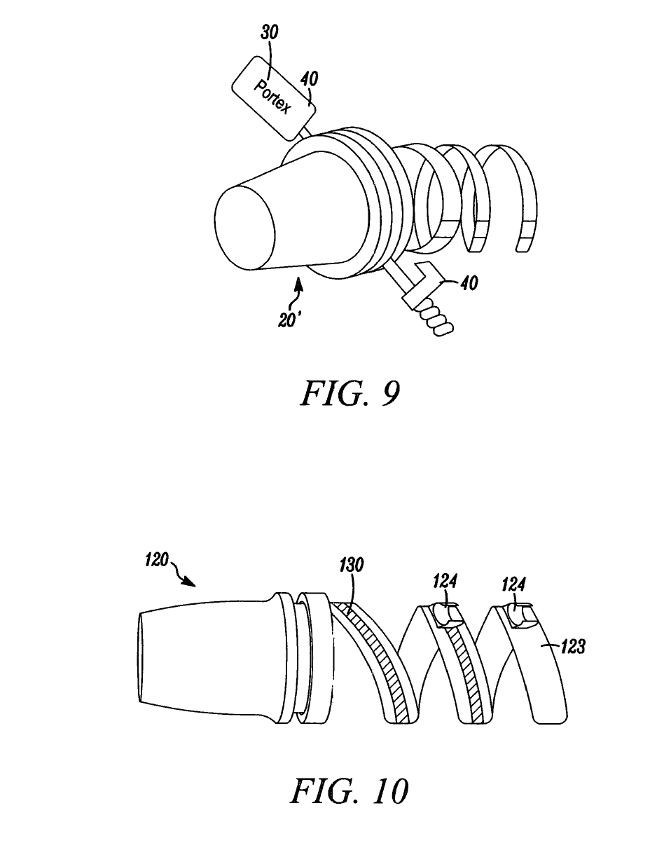

FIG. 9 is a perspective view of another form of machine end component; and

FIG. 10 is a side elevation view of further alternative form of machine end component including a metal reinforcing member.

With reference first to FIGS. 1 to 3 there is shown a tracheostomy tube having a tubular shaft 1 with substantially straight patient and machine end portions 2 and 3 linked by a curved portion 4. The machine end 5 of the tube is terminated by a coupling 6 having a male luer taper and a flange 7 extending radially outwards from the coupling. The flange 7 has openings 8 close to opposite ends for use in securing a tape or the like by which the tube is fastened about the patient's neck. Towards the patient end 10 of the tube an inflatable sealing cuff 11 extends around the shaft 1, the cuff being inflated or deflated via an inflation line 12 extending along the shaft and through the flange 7. The inflation line 12 is terminated by a conventional combined inflation indicator and valved connector 13.

As so far described the tube is conventional. The tube is installed, in use, by inserting its patient end 10 through a surgically-made opening in the neck so that the patient end locates in the trachea and the machine end 5 and flange 7 lie adjacent the skin on the outside of the patient. The sealing cuff 11 is inflated via the connector 13 and inflation line 12 so that the cuff seals with the trachea and ventilation gas supplied to and from the respiratory passages is confined to flow along the bore 9 of the tube.

The tube differs from previous tubes by being formed from at least two materials of differing hardness.

The tube includes a first, machine end component 20, shown in more detail in FIG. 3, that is moulded of a relatively hard plastics material, such as acetal, polycarbonate, acrylic, polypropylene, rigid PVC, polyimide, polyamide, sytrenic plastics including SAN, ABS, polyurethane or the like. These, or other materials, could be further reinforced by incorporating other materials such as fillers or fibres, which could be of glass bead, glass fibre, carbon fibre, Kevlar aramid or the like. Metal reinforcement could be used such as in wire or strip form and could be of stainless steel or Nitinol. Where a metal reinforcement is used this may be confined to a localised region of the component excluding the coupling. The component 20 comprises the coupling 6 with a short boss 21 at the forward, patient end of the coupling and having an annular groove 22 around its outside. Reinforcing means in the form of a reinforcement member 23 extends forwardly from the boss 21. The reinforcement member 23 takes the form of a strip extending along a helical path along the tube. The reinforcement member 23 extends preferably along at least that part of the tube extending through the tracheostomy opening or stoma so as to reinforce this part of the tube and protect it from pressure applied by the surrounding tissue. Each turn of the reinforcement strip 23 includes an optional moulded clip 24 of horse-shoe section on its outer surface and aligned with one another so that the inflation line 12 can be retained by the clips during the moulding process. Alternatively, the inflation line could be provided in a conventional manner, such as by attachment to the outer surface of the tube shaft after moulding. The reinforcement member could also include clips or other formations (not shown) by which the machine end component 20 is retained in a mould to hold it in place during the moulding process. Instead of moulding the reinforcing member 23 in a helical shape it could be moulded straight and subsequently formed into a helical shape, such as by heat treatment.

The remainder of the tube is provided by a second component 29 formed by overmoulding a plastics material onto the machine end component 20. More particularly, the machine end component 20 is placed in an injection mould (not shown) with its boss 21, the reinforcing member 23 and inflation line 12 being located in the mould cavity, which defines the internal and external shape of the tube shaft 1. A plastics material 26 is injected into the cavity and is overmoulded on the exposed parts of the machine end component 20. The injected plastics material 26 is chosen to be softer than the material of the machine end component and may, for example, be a silicone material, a thermoplastic, such as non- phthalate PVC or other materials such as thermoplastic rubber. The softer material 26 could be of the same type of plastic as that forming the machine end component 20 but of a softer grade. The cavity of the mould also defines the shape of the flange 7 so that the injected plastics material forms the flange on the groove 22. After the tube has been removed from the mould the cuff 11 it is attached to the shaft 1 in communication with the patient end of the inflation line 12, the opposite end of the inflation line being connected to the connector 13. The inflation line connector 13 could instead be attached with the inflation line 12 before the moulding operation to remove a post-moulding process step. The reinforcing member 23 could be completely encapsulated within the wall thickness of the overmoulded material 26 forming the shaft 1 or it could lie along the outer or inner surface of the shaft.

The completed tube, therefore, has a relatively soft, flexible patient end portion 2 and intermediate portion 4 provided entirely by the overmoulded softer plastics material 26 so that it flexes readily to the shape of the anatomy and to accommodate any changes in shape, such as caused by movement of the patient. It also reduces the risk of trauma to the lining of the trachea caused by any rubbing of the patient end tip 10 of the tube during insertion. The region of the tube extending through the neck tissue is formed of a combination of the softer overmoulded plastics material 26 and the relatively hard reinforcement member 23 so that this region is stiffer than the patient end 10 and more able to resist radial crushing forces applied by the neck tissue. This region of the tube, therefore, has the desired attributes to ensure that the tube is not unduly deformed, thereby ensuring that the bore 9 remains open to allow free flow of gas and to allow any tubes, probes or the like to be freely inserted along the bore. The flange 7 may be formed entirely of the softer plastics material to ensure that it readily bends to follow the anatomy of the neck when it is secured and without causing chafing or trauma to the skin. The machine end coupling 6 is formed entirely of the harder plastics material so that it is rigid and maintains an effective seal with any mating coupling attached with it.

The combination of the softer overmoulded plastics material 26 with the harder plastics moulding 20 enables the tube shaft 1 to be given the desired mechanical properties in the desired regions without increasing the wall thickness of the shaft. A tube formed entirely of the softer plastics material could be made more resistant to crushing by increasing its wall thickness but this would result in a narrower bore and hence reduce gas flow and increase in the pressure drop along the tube. The smaller bore diameter could also increase the risk of occlusion from accumulated secretions. The construction of the present invention, by contrast, enables a relatively high ratio of outside diameter to bore diameter. The relatively large internal diameter allows an inner cannula (not shown) with a relatively large diameter to be used. The inner cannula and the softer plastics of the tube shaft could both be thermo softening so that the combination of the inner cannula and outer tube is also thermo softening.

Although the reinforcing member is described as extending only along that part of the tube shaft extending through the tracheostomy stoma, it would be possible to extend it further if additional radial strength were required further along the tube. It might be advantageous, for example, to provide reinforcement within the region of the tube shaft under the sealing cuff because, when this is inflated against the tracheal wall it could, if not reinforced, apply pressure to the tube shaft sufficient to deform it and provide an obstruction within the bore. If an inner cannula were used this pressure could cause the tube shaft to grip the outside of the inner cannula and make it difficult to withdraw. The reinforcing member could be shaped to vary its reinforcing effect such as by varying the pitch of the helix and, or alternatively, varying the thickness or width of the member. FIG. 4, for example, shows a reinforcing member 23′formed with short regions 30 where it is thinner and weaker.

The reinforcement means need not be provided by a member of helical shape but could take other forms. For example, the reinforcement member could be provided by one or more longitudinal spines supporting one or more circular or part-circular annular ribs. FIGS. 5 and 6 show an arrangement where the reinforcing means is provided by four parallel, longitudinally-extending spines 23″.

The tube could additionally include provision for suction above the cuff as shown in FIGS. 7 and 8. In this arrangement the reinforcing member includes two sets of clips 24 and 24′ along opposite sides of the helix into which the cuff inflation line 12 and the suction line 31 respectively can be clipped. If either the inflation line or the suction line is deformed by the pressure of the overmoulding process they could be reinforced during assembly with a rigid wire of a metal or plastics extended along their bores, which is removed after completion of the moulding process.

Identification features 30 on the flange 7, such as manufacturer name, size information, product code or other legend or colour coding could be formed on tabs 40 attached as a part of the machine end component 20′, as shown in FIG. 9, so that they are overmoulded and encapsulated by the softer plastics material 26 during the moulding process. The legends would be visible through overmoulded material 26. This would have the advantage of avoiding the need for any subsequent printing process and also avoid the need for an engraved surface on the flange, thereby avoiding any potential dirt trap.

The helical part of the machine end component 120, shown in FIG. 10, includes a plastics spiral reinforcement member 123 with an additional metal reinforcing strip 130 attached with it along its length by means of clips 124 moulded with the plastics member. Alternatively, the plastics member 123 could be overmoulded about the metal reinforcing member 130.

The harder component need not be moulded to shape but could instead, for example, be provided by a tape, wrapping this helically around a mandrel and then heat treating this to set it to the desired helical shape.

The present invention enables a tube to be provided that has both the desired strength characteristics where this is needed and a soft flexible patient end. The same tube could also be used with an inner cannula. This reduces the need for multiple separate tubes in different situations. The invention could enable a tube with a larger bore so as to satisfy the majority of patient populations. The thinner wall possible with the present invention could allow a size 8 bore with a size 7 outside diameter, thereby reducing the number of tube sizes needed and consequently reducing the amount of tube stocks a hospital needs to keep.

The invention is not confined to tracheostomy tubes but may have application to other tubes where it is desirable to reinforce a part of the length of the tube extending through a body opening.

1-10. (canceled)

11. A medico-surgical tube having a coupling at its machine end and a shaft adapted to extend through an opening into a body cavity, characterised in that the shaft is formed of at least first and second materials of different hardness, that the first material is harder than the second material, that the coupling is formed primarily of the first material, and that the first material of the coupling and of the shaft are formed as one component.

12. A tube according to claim 11, characterised in that the region of the shaft extending within the opening has a greater proportion of the first material to the second material than the region of the shaft extending within the body cavity such that the region of the shaft extending within the opening is more rigid than the region of the shaft extending within the body cavity,

13. A tube according to claim 11, characterised in that the tube has an elongate member of the first material extending along the region of the tube extending within the opening, and that the elongate member is embedded in the second material.

14. A tube according to claim 13, characterised in that the elongate member extends around and along the region within the opening.

15. A tube according to claim 14, characterised in that the elongate member is of helical shape.

16. A tube according to claim 11, characterised in that the tube has a flange formed by a member of the first material and an overmoulding of the second material.

17. A tube according to claim 16, characterised in that the member of the first material includes markings visible through the second material.

18. A tracheostomy tube formed of a first, relatively hard material and a second, relatively soft material, characterised on that the tube is formed of a first component of the relatively hard material providing both a machine end coupling at a machine end of the tube and a reinforcing member extending along a part at least of the length of a shaft, that the shaft has a portion extending through neck tissue and formed of a combination of the first and second materials and that the shaft has a patient end portion extending within the trachea and formed substantially entirely of the second, relatively soft material.

19. A tube according to claim 18, characterised in that both the first and second materials are plastics materials.

20. A tube according to claim 19, characterised in that the first and second materials are different grades of the same type of plastics.

21. A tube according to claim 11, characterised in that both the first and second materials are plastics materials.
